(19) United States
(12) Patent Application Publication
(10) Pub. No.: US 2009/0187129 A1
(43) Pub. Date: Jul. 23, 2009

(54) Trauma cervical stability device and methods of using same for diagnostic purposes

(76) Inventors: Peleg Ben-Galim, Houston, TX (US); Seyed B. Kalantar, Houston, TX (US); John A. Hipp, Manvel, TX (US); Charles A. Reitman, Bellaire, TX (US)

Correspondence Address: GREENBERG TRAURIG (HOU);INTELLECTUAL PROPERTY DEPARTMENT, 1000 Louisiana Street, Suite 1800, Houston, TX, 77002, US

(21) Appl. No.: 12/009,653

(22) Filed: Jan. 22, 2008

(51) Int. Cl. A61F 5/00; A61G 15/00

(52) U.S. Cl. 602/18; 602/17; 602/36; 128/845

(57) ABSTRACT

Trauma cervical stability devices for using by ambulatory personnel arriving at the scene of an injured patient are disclosed. The trauma cervical stability devices comprise a cap element, releasable and adjustable head straps, a shoulder harness, and at least one adjustable member operatively connected to the cap element and the shoulder harness. The trauma cervical stability devices are compact, easy to use, inexpensive to manufacture, and can be placed on a patient with little or no movement of the patient. The trauma cervical stability devices are also useful in diagnosing the severity of damage to a neck and the stability of the patient's neck by applying forces to the patient's head using the trauma cervical stability device.

### BACKGROUND

1. Field of Invention

The invention is directed to trauma cervical stability devices and, in particular, to adjustable cervical stability devices capable of easy and cost effective use by ambulatory personnel at the scene of the injury and of allowing injury diagnosis upon arrival at the hospital.

2. Description of Art

Trauma cervical collars are generally known in the art. Briefly, these cervical collars are carried on ambulances and other emergency personnel vehicles and are usually one-time use devices. These cervical collars provide limited, if any, means to adjust the cervical collar to fit the patient while securing the cervical collar to the injured patient. Generally, the patient must be moved to secure the cervical collar to the patient. Movement of the patient, however, can cause additional injury to the patient. In those cervical collars where adjustment is provided, the adjustment capabilities are limited which can result in the patient's head not being sufficiently stabilized with respect to the patient's spine, neck, or body.

In other cervical collars, adjustment of the cervical collar may be achieved without excessive movement of the patient, however, the cervical collar is large and complex. Thus, these cervical collars are not only difficult to store in emergency vehicles where space is limited, they are difficult to use by emergency personnel. Accordingly, these types of devices instead are used to rehabilitate the patient's injured neck, e.g., after diagnosis and, generally, operation on the patient at a hospital, as opposed to stability a traumatic injury to a patient at the scene of the injury.

### SUMMARY OF INVENTION

Trauma cervical stability devices for using by ambulatory personnel arriving at the scene of an injured patient are disclosed. Broadly, the trauma cervical stability devices comprise a cap element, releasable and adjustable head straps, a shoulder harness, and at least one adjustable member operatively connected to the cap element and the shoulder harness. The trauma cervical stability devices are compact, easy to use, inexpensive to manufacture, and can be placed on a patient with little or no movement of the patient. The trauma cervical stability devices are also useful in diagnosing the severity of damage to a neck and the stability of the patient's neck by applying forces to the patient's head using the trauma cervical stability device. It is to be understood, however, that the effects and results of the trauma cervical stability devices disclosed herein are dependent upon the skill and training of the operators and surgeons.

### BRIEF DESCRIPTION OF DRAWINGS

FIG. 1 is a perspective view of one embodiment of the trauma cervical stability device disclosed herein shown secured to a patient.

FIG. 2 is a perspective view of the trauma cervical stability device shown in FIG. 1 illustrated in the flat position before being secured to a patient.

FIG. 3 is a front perspective view of another embodiment of the trauma cervical stability device disclosed herein shown secured to a patient.

FIG. 4 is a side perspective view of the trauma cervical stability device illustrated in FIG. 3 shown secured to a patient.

FIG. 5 is a back perspective view of the trauma cervical stability device illustrated in FIG. 3 shown secured to a patient.

FIG. 6 is a close-up perspective view of a track for use with the trauma cervical stability device shown in FIG. 3.

FIG. 7 is a cross-sectional view of the track shown in FIG. 6.

FIG. 8 is a graph showing sagittal plane intervertebral translation at C4-C5 in intact spines.

FIG. 9 is a graph showing change in average disc space height (% C4 endplate width) per 100N applied traction.

FIG. 10 is a graph showing the change in average disc space height with applied traction (% C4 endplate width).

FIG. 11 is a graph showing applied traction to the head (N) required to distract disc space 1 mm.

While the invention will be described in connection with the preferred embodiments, it will be understood that it is not intended to limit the invention to that embodiment. On the contrary, it is intended to cover all alternatives, modifications, and equivalents, as may be included within the spirit and scope of the invention as defined by the appended claims.

### DETAILED DESCRIPTION OF INVENTION

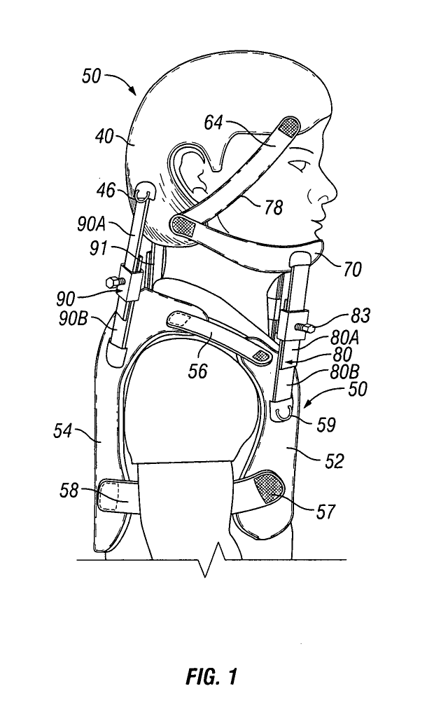

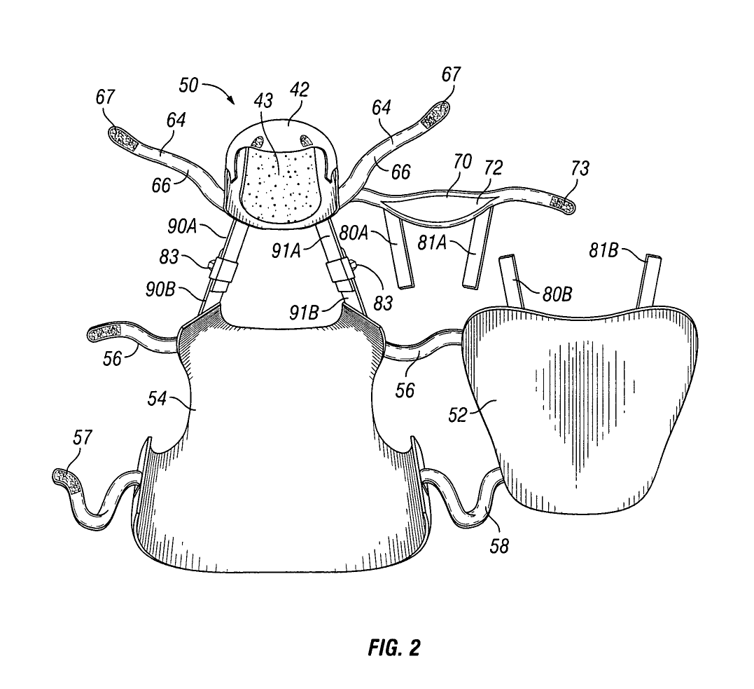

Referring now to FIGS. 1-2, in one embodiment, trauma cervical stability device 30 includes cap element 40, shoulder harness 50, head strap 64, chin strap 70, anterior adjustable members 80, 81 and posterior adjustable members 90, 91. Cap element 40 comprises an inner wall surface 42 (FIG. 2) shaped for receiving the head of a person or patient. Cap element 40 may be formed from any suitable material that provides rigidity, such as plastic materials. As shown in FIG. 2, inner wall surface 42 includes a cushion material 43, such as foam, so that inner wall surface 42 can conform to the contour of the patient's head.

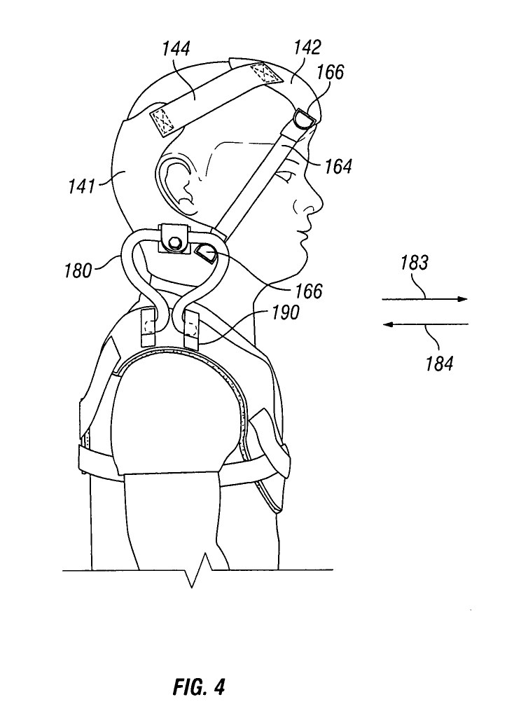

Cap element 40 covers the posterior and crown or top portions of the head of the patient. In the embodiment shown in FIGS. 1-2, cap element 40 covers not only the posterior and crown portions of the head of the patient, but also extends over the forehead of the patient. Although cap element 40 is shown in the embodiment of FIGS. 1-2 as being formed of a single piece of material, it is to be understood that cap element 40 may be formed by two or more separate pieces such as in the embodiment of FIGS. 4-7.

Due to cap element 40 covering the posterior surface of the patient's head as well as a at least a portion of the frontal lobe of the patient's head, which, in some embodiments also includes covering a portion of the forehead of the patient, when cap element 40 is connected to shoulder harness 50 as discussed in greater detail below, a downward force is applied to the head of the patient to assist in stabilizing the head of the patient relative to the body of the patient. The term “downward force” is used herein to describe forces applied in the direction of from the top of the head to the body and includes forces applied straight down toward the body, e.g., at a vertical angle (i.e., at a right angle to the horizon), as well as at an angle other than a vertical angle, e.g., at a 45 degree angle, a 30 degree angle, a 10 degree angle, an 80 degree angle, to the vertical angle.

Shoulder harness 50 includes front or breast plate 52 and back plate 54. One or both of breast plate 52 and back plate 54 includes inner wall surfaces having a cushion for conforming to the shape of the patient's body to support and comfort the patient's body. In one embodiment, both breast plate 52 and back plate 54 are formed from a rigid material, such as plastic, having a foam insert secured to the inner wall surface of the breast plate 52 and back plate 54. Shoulder straps 56 and body straps 58 releasably secure breast plate 52 with back plate 54. In the embodiment shown in FIGS. 1-2, shoulder straps 56 and body straps 58 include Velcro® pads 57 to releasably secure breast plate 52 to back plate 54.

Head strap 64 and chin strap 70 include a soft, cushioned inner wall surfaces 66, 72, respectively for conforming to and/or providing comfort to, the patient's head and chin. Head strap 64 and chin strap 70 are releasably secured to cap element 40. In the embodiment shown in FIGS. 1-2, head strap 64 and chin strap 70 include Velcro® pads 67, 73 to releasably secure head strap 64 and chin strap 70 to cap element 40.

Anterior adjustable members 80, 81 are secured at their upper and lower ends to chin strap 70 and breast plate 52, respectively. In one embodiment, anterior adjustable members 80, 81 are secured at their upper and lower ends to chin strap 70 and breast plate 52 respectively by rotatable members (not shown) to allow the connections between the upper and lower ends of anterior adjustable members 80, 81 to chin strap 70 and breast plate 52, respectively, to pivot and rotate so that the angle of intersection between anterior adjustable members 80, 81 chin strap 70 and breast plate 52 can be adjusted. Suitable rotatable members include, but are not limited to, lockable ball and socket connections so that the connections can pivot to the desired orientation and locked in place. Alternatively, only one of the connections between anterior adjustable members 80, 81 and chin strap 70 or breast plate 52 is rotatable, so that the other connection is fixed, i.e., the angle of intersection between anterior adjustable members 80, 81 and chin strap 70 or breast plate 52 cannot be adjusted.

Posterior adjustable members 90, 91 are secured at their upper and lower ends to cap element 40 and back plate 54, respectively. In one embodiment, posterior adjustable members 90, 91 are secured at their upper and lower ends to cap element 40 and back plate 54 respectively by rotatable members (not shown) to allow the connections between the upper and lower ends of posterior adjustable members 90, 91 to cap element 40 and back plate 54, respectively, to pivot and rotate so that the angle of intersection between posterior adjustable members 90, 91 and cap element 40 and back plate 54 can be adjusted. Suitable rotatable members include, but are not limited to, ball and socket connections. Alternatively, only one of the connections between posterior adjustable members 90, 91 and cap element 40 or back plate 54 is rotatable, so that the other connection is fixed, i.e., the angle of intersection between posterior adjustable members 90, 91 and cap element 40 or back plate 54 cannot be adjusted.

Anterior adjustable members 80, 81 and posterior adjustable members 90, 91 may be any device known to persons skilled in the art that are capable of having their length adjusted. As shown in FIGS. 1-2, both anterior adjustable members 80, 81 and posterior adjustable members 90, 91 are formed by upper members 80A, 90A, and lower members 80B, 90B in sliding engagement with each other and held in contact with each other by bracket 82 having set screw 83. Tightening set screw 83 secures upper members 80A, 90A, and lower members 80B, 90B within bracket 82 so that no additional lengthening of anterior adjustable members 80, 81 or posterior adjustable members 90, 91 is permitted. Loosening set screw 83 releases upper members 80A, 90A, and lower members 80B, 90B from within bracket 82 so that they can sliding axially along each other thereby permitting additional lengthening of anterior adjustable members 80, 81 and posterior adjustable members 90, 91.

One or more attachment members may be included as part of trauma cervical stability device 30 so that pulleys, weights, loads, or forces can be applied to trauma cervical stability device 30 in one or more directions. For example, cap element attachment member 46 may be included as part of cap element. As shown in FIG. 1, cap element attachment member 46 is located at the upper end of posterior adjustment member 90. Additionally, breast plate attachment member 59 is located at the lower end of anterior adjustment member 80. Attachment members 46, 59 are shown in FIGS. 1-2 as hooks, however, it is to be understood that attachment members 46, 59 may be any other device capable of securing pulleys or other traction or loads to trauma cervical stability device 30. Suitable attachment members 46, 59 include snaps and belt and buckle connections.

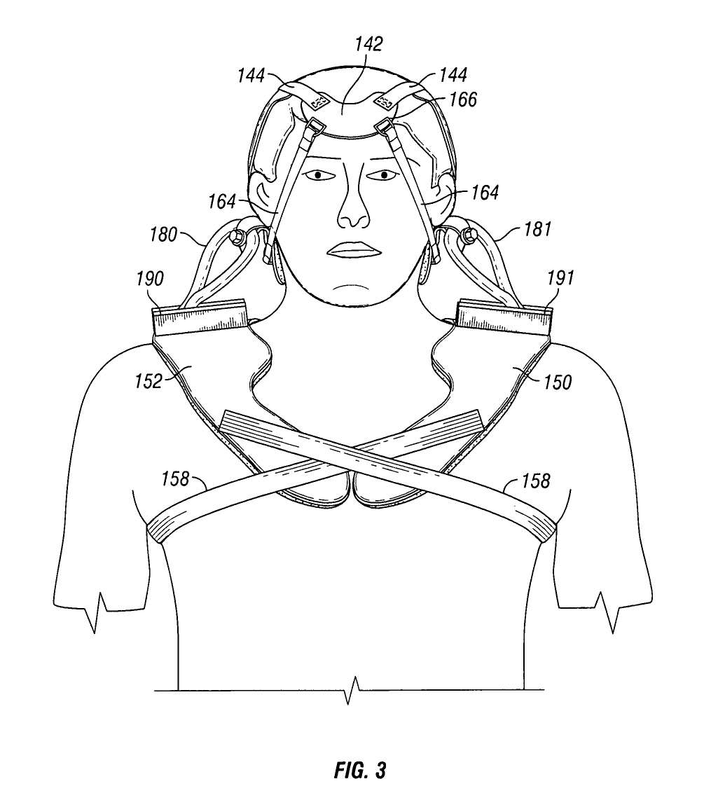

Referring now to FIGS. 3-7, in another embodiment, trauma cervical stability device 130 includes cap element 140, shoulder harness 150, head straps 164, and adjustable members 180 and 181. Cap element 140 comprises two portions, posterior portion 141 and anterior portion 142. As shown in FIGS. 3-5, anterior portion 142 is position above the patient's forehead. Posterior portion 141 is connected to anterior portion 142 by cap element straps 144. Like the embodiment of FIGS. 1-2, an inner wall surface of one or both of posterior portion 141 and anterior portion 142 may be shaped for receiving the head of the patient and cap element 140 may be formed from any suitable material that provides rigidity, such as plastic materials. Additionally, a cushion material such as foam may be disposed on the inner wall surfaces of one or both of posterior portion 141 and anterior portion 142 so that inner wall surfaces of these portions of cap element 141 can conform to the contour of the patient's head. Medical gauze may also be placed between the patient's head and cap element 140 to help control bleeding from lacerations on the head. The pressure from cap element 140 can be used to help control bleeding from head lacerations.

Cap element 140 covers the posterior and crown or top portions of the head of the patient. Due to cap element 140 covering the posterior surface of the patient's head as well as a at least a portion of the crown portion of the patient's head, which, in some embodiments also includes covering a portion of the forehead of the patient, when cap element 140 is connected to shoulder harness 150 as discussed in greater detail below, a downward force is applied to the head of the patient to assist in stabilizing the head of the patient relative to the body of the patient. The term “downward force” has the same meaning as described above with respect to the embodiment of FIGS. 1-2.

Shoulder harness 150 includes front or breast plate 152 and, optionally, back plate 154. One or both of breast plate 152 and back plate 154 includes an inner wall surface having a cushion for conforming to the shape of the patient's body to support and comfort the patient's body. In one embodiment, both breast plate 152 and back plate 154 are formed from a rigid material, such as plastic, having a foam insert secured to the inner wall surface of the breast plate 152 and back plate 154. Back plate straps 156 and body straps 158 releasably and adjustably secure breast plate 152 with back plate 154 such as through the use of Velcro® pads, buckles, snaps, stitching, or other fastener members (not shown). Body straps 158 can be directly connected from the front of breast plate 152, around the body, and back to breast plate 152, such as to the portion of breast plate 152 that rests on the back of the patient's shoulders. Thus, back plate 154 is not required. Body straps 158 can be releasably and adjustably connected to front plate 152, back plate 154 or front and back plates 152, 154 to facilitate securing trauma cervical stability device 130 to the patient.

Cap element straps 144 and head straps 164 can include soft, cushioned inner wall surfaces for conforming to and/or providing comfort to, the patient's head. Both cap element straps 144 and head straps 164 may be releasably and adjustably connected to cap element 40 such as through the use of Velcro® pads, buckles, snaps, stitching, or other fastener members (not shown). Cap element straps 144 can be releasably and adjustably connected to one or both of posterior portion 141 and/or anterior portion 142 of cap element 141. Head straps 164 can be releasably and adjustably connected to cap element 140 at both ends of head straps 164. As shown in FIGS. 3-5, head straps 164 are releasably and adjustably connected to posterior portion 141 and anterior portion 142 of cap element 140 at both ends of head straps 164 by buckles 166.

Adjustable members 180, 181 are secured to cap element 140. In the embodiment of FIGS. 3-7, adjustable members 180, 181 are secured to posterior portion 141 of cap element 140. The connection between adjustable members 180, 181 and cap element 140 can comprise a rotatable member to provide a pivot point and an adjustable fastener such as a set screw or wing-nut.

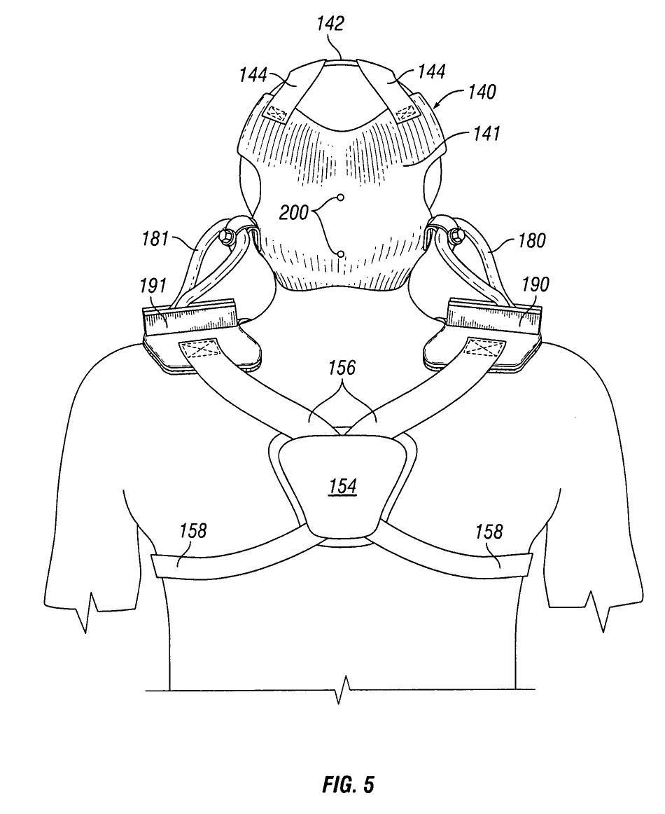

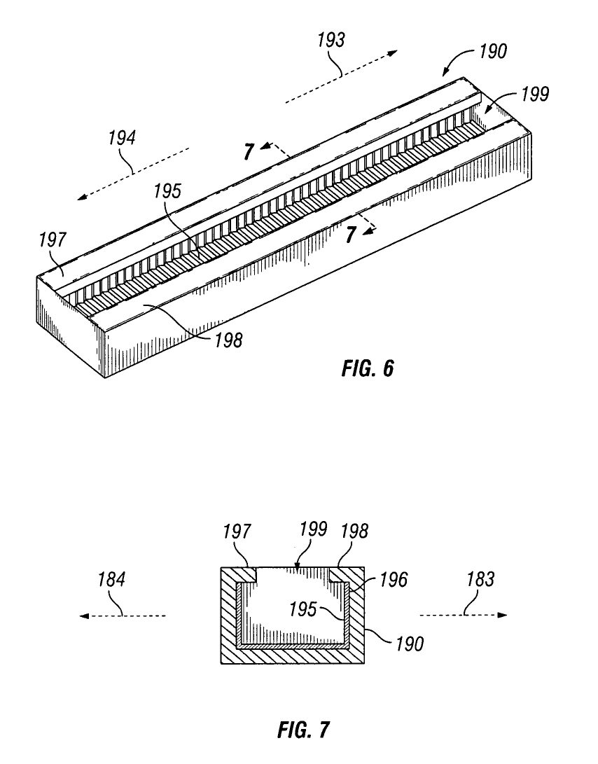

The lower ends of adjustable members 180, 181 are operatively disposed in tracks 190, 191, respectively. Tracks 190, 191 permit movement of the lower ends of adjustable members 180, 181 in the direction of arrows 193 (i.e., toward the patient's head), 194 (i.e., away from the patient's head) (FIG. 6). The lower ends of adjustable members 180, 181 may be operatively associated with tracks 190, 191 in any manner known to persons of ordinary skill in the art so as to provide movement in the direction of arrows 193, 194.

Referring now to FIGS. 6-7, in one particular embodiment, track 190, which for purposes of this embodiment is identical to track 191, comprises ratchet profile 195 disposed along inner wall surface 196 of tracks 190. Ratchet profile 195 permits movement of the lower ends of adjustable members 180, 181 in one direction, i.e., in the direction of arrow 193 toward the head, so that the orientation of adjustable members 180, 181 can be modified as necessary to secure trauma cervical stability device 130 to the patient. To move the adjustment members 180, 181 away from the head, each adjustment member 180, 181 or each track 190, 191 may include a release member, discussed in greater detail below, that releases adjustment members 180, 181 from ratchet profile 195, allowing movement of the lower ends of adjustment members 180, 181 within track 190, 191, respectively.

The upper side of track 191 comprises rails 197, 198 and slit 199. Rails 197, 198 restrict the lower ends of adjustable members 180, 181 from being disconnected from tracks 190, 191, respectively, while slit 199 permits the lower ends of adjustable members 180, 181 to be inserted into, and made operatively associated with, tracks 190, 191, respectively.

In this particular embodiment, the lower ends of adjustable members 180, 181 comprise a front end and a back end, each of which is outwardly biased. In other words, both front end and back ends are designed such that the front end exerts a force in the direction of arrow 183 (FIGS. 4 and 7) and the back end exerts a force in the direction of arrow 184 (FIGS. 4 and 7). Therefore, to move lower ends of adjustable members 180, 181 along tracks 190, 191 respectively, away from the head of the patient (i.e., in the direction of arrow 194), front and back ends are pinched together so as to disengage from ratchet profile 195. Thus, in this embodiment, the compression of the lower ends of adjustable members 180, 181 is the release member mentioned above. The lower ends of adjustable members 180, 181 can then be moved along the length of tracks 190, 191, respectively, in the direction away from the head to adjust the fit of trauma cervical stability device 130 to the patient.

To initially connect adjustable members 180, 181 to tracks 190, 191, adjustable members 180, 181 are disposed through slit 199 within tracks 190, 191, respectively, by turning adjustable members 180, 181 approximately 90 degrees from the orientation shown in FIGS. 3-5. After the lower ends are within tracks 190, 191, adjustable members 180, 181 are rotated 90 degrees so that the lower ends of adjustable members 180, 181 are disposed under rails 197, 199. Due to the outward biases of the front and back ends of each adjustable members 180, 181, the lower ends of adjustable members 180, 181 move outwardly and under rails 197, 199. As a result, the lower ends are retained within tracks 190, 191.

In another specific embodiment, cap element 140 includes one or more metallic studs 200 (FIG. 5). These studs are disposed substantially along the axis of the vertebra so as to provide an alignment point for imaging, e.g., X-ray, purposes. Further, attachment members (not shown) can be included as part of trauma cervical stability device 130 to provide the same functions as attachment members 46, 59 in the embodiment shown in FIGS. 1-2.

The embodiment shown in FIGS. 3-7 operates and provides the same functionality as the embodiment shown in FIGS. 1-2, with the exception of the specific methods of how trauma cervical stability device 130 is installed and adjusted on the patient. These differences are evident to persons skilled in the art based upon the discussed above with respect to the differing structures.

Although all of the structures of the trauma cervical stability devices disclosed herein can be formed out of any desired or necessary material to provide the required rigidity, plastic materials and other similar materials do not interfere with X-rays and other non-invasive imaging devices so that the trauma cervical stability devices are not required to be removed prior to imaging the patient's injury.

Trauma cervical stability devices 30, 130 may be used in any number of diagnostic techniques. In one such use, the trauma cervical stability device diagnoses the severity of damage to the neck of patient as well as diagnose whether the neck is stable prior to administering additional aid to the patient. In one embodiment, the trauma cervical stability device is secured to a patient's body and head by placing the back plate on the posterior side of the patient and the cap element on the posterior surface of the head of the patient. The breast plate is then placed on the anterior side of the patient and the one or more head straps are secured along the sides of the head of the patient and, if included, the chin strap is secured under the chin of the patient. The back plate is secured to the breast plate through the body straps and, if present, the shoulder straps.

After securing the trauma cervical stability device to the patient, each of the adjustable members are manipulated, e.g., extended, retracted, rotated, tilted, etc., to conform the trauma cervical stability device to the patient's neck and body orientation at the scene of the injury. After manipulating the adjustable member(s), the patient's neck is stabilized relative to the patient's body.

Although the patient's neck is “stabilized” relative to the body through the trauma cervical stability device, it is to be understood that the patient's neck may not be stable without the trauma cervical stability device. Additionally, the patient's neck may have sustained substantially damage that may not be evident due to the trauma cervical stability device being secured to the patient's head and body. Therefore, as discussed below, the trauma cervical stability device can be further manipulated by a physician at the hospital to determine whether the neck of the patient is stable and, if not stable, how severe the damage to the patient's neck might be.

To facilitate application of controlled traction loads to the head using the trauma cervical stability devices disclosed herein, a simple load sensing mechanism can be integrated into the articulation between adjustable members 180, 181 and cap element 140. This load-sensing articulation can provide instant feedback to a physician regarding the relative magnitude of traction that is being applied to the head by the stabilization device.

In the embodiment in which the physician determines whether the patient's neck is stable, the physician places a force or a load onto the patient's head and/or body such as by securing known weights to the attachment members of the trauma cervical stability device. The force or load caused by the weights is directed in a known direction using a pulley system. For example, the physician may place a load of 20 pounds in the upward direction parallel to the spine, i.e., pulling up on the head of a patient away from the body. If the motion between vertebrae in the spine is more than the intervertebral motion that occurs for an uninjured patient, the physician knows that the patient's cervical spine is not stable and that further diagnostic and imaging techniques, such as an MRI, are needed.

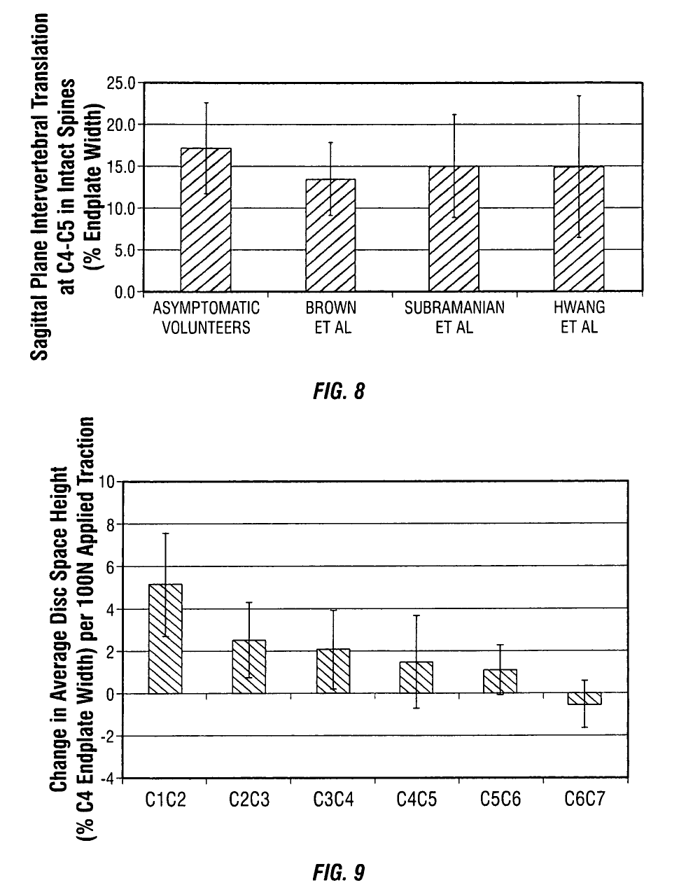

Using trauma cervical stability device 130, the inventors have completed a series of studies using whole cadavers to determine how best to diagnose injuries to the cervical spine. The whole cadaver model is a very good representation of motion live humans, since intervertebral motion in the fresh, unembalmed cadavers was statistically equivalent to motion that the authors have documented in live, asymptomatic humans. The equivalence of motion in fresh cadavers versus live humans is illustrated in FIG. 8. In FIG. 8, the data for the asymptomatic volunteers identified is from Reitman C. A., Mauro K. M., Nguyen L. et al., Intervertebral motion between flexion and extension in asymptomatic individuals; Spine 2004; 24:2832-43, which is hereby incorporated by reference in its entirety; the data designated “Brown, et al.” is from Brown T., Reitman C. A., Nguyen L., et al., Intervertebral motion after incremental damage to the posterior structures of the cervical spine; Spine 2005; 30:E503-E508, which is hereby incorporated by reference in its entirety; the data designated “Subramanian et al.” is from Subramanian N., Reitman C. A., Nguyen L., et al., Radiographic assessment and quantitative motion analysis of the cervical spine after serial sectioning of the anterior ligamentous structures; Spine 2007; 32:518-26, which is hereby incorporated by reference in its entirety; and the data designated “Hwang et al” is from Hwang H., Hipp J. A., Ben-Galim P., et al., Threshold cervical range-of-motion neccesary to detect abnormal intervertebral in cervical spine radiographs; Spine 2007 (currently in Press), which is hereby incorporated by reference in its entirety.

During one study, traction loads were applied to the heads of whole cadavers before and after creating injuries to the cervical spine. These experiments defined the loads that need to be applied to the head to diagnose an injury to the spine. These experiments also defined the level of loads that will not overly distract the spine yet will allow detection of damage to the spine. Results of these studies are shown in FIG. 9 which illustrates the amount of distraction that occurs in the intact cervical spine with application of axial traction, for each intervertebral level in the cervical spine.

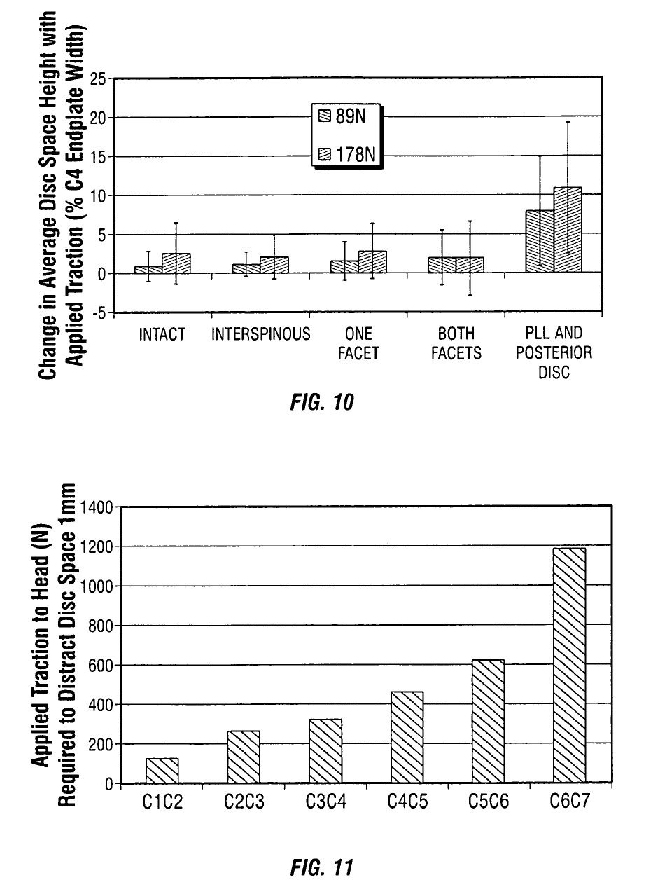

Referring now to FIG. 10, additional results from the study using whole human cadavers are shown. As illustrated in FIG. 10, there is not a very large amount of separation between vertebrae in response to traction loads applied to the head until extensive damage is done to the spine. Statistical analysis of this data also show that a modest traction load (89 Newton=20 lbs) is actually more sensitive for diagnosing cervical injuries than a higher load (178 Newton=40 lbs).

It was further determined from the whole cadaver studies that much less traction is needed to distract the upper cervical versus lower cervical vertebrae. This observation is illustrate in FIG. 11. Using these results of these studies, physicians using trauma cervical stability device 130 can apply a number of different loads to the spine and, depending on the movement of the vertebra, can diagnose the severity of neck injury. For example, a low load would first be used to identify potential upper cervical injuries, followed by a modest load to diagnose upper or middle cervical spine injuries, followed by a higher load that would uncover injuries at any level.

In addition to the physician determining whether the neck of the patient is stable, the physician can also engage in additional diagnostic investigation as to the severity of the patient's injured and unstable neck. To do so, the physician applies known forces or loads onto the patient's head and/or body in the same manner as discussed above and then measures the distance or amount of movement between vertebrae in the spine in each direction of the force or load. Intervertebral motion is measured from x-rays or other imaging methods or devices taken before and after the load is applied. The physician then compares each of the measured intervertebral motions to motions that are indicative of certain injuries. For example, if the two vertebrae rotate away from each other when 20 pounds of force is exerted on the patient's head in the upward direction parallel to the spine, i.e., pulling up on the head of a patient away from the body, then the physician can be fairly confident that the patient's injury is extremely severe. If the two vertebrae rotate in a manner resembling motion during flexion of the head and neck, this type of rotation suggests injury to posterior structures of the spine, such as the interspinous ligaments, facets, and/or ligamentum flavum. If, during application of axial traction, the two vertebrae rotate in a manner resembling motion that occurs during extension, this type of rotation suggests damage to anterior structures, such as the anterior longitudinal ligament and/or the intervertebral disc.

It is to be understood that the invention is not limited to the exact details of construction, operation, exact materials, or embodiments shown and described, as modifications and equivalents will be apparent to one skilled in the art. For example, the head straps may be a single strap that extends from one side of the cap element, passes through a slot on the top of the cap element, and extends to the other side of the cap element where it is releasably and adjustably connected to the cap element. Moreover, the tracks may not include a ratchet profile, but instead include slots or holes into which the lower ends of the adjustable elements are inserted. Accordingly, the invention is therefore to be limited only by the scope of the appended claims.

What is claimed is:

1. A trauma cervical stability device for use on a patient having a body and a head with a posterior surface and a crown surface, the trauma cervical stability device comprising:
a cap element, the cap element having an inner wall surface for contacting the head of the patient, the inner wall surface having a contacting surface area between the inner wall surface and the head of the patient sufficient to contact the posterior surface and the crown surface of the head of the patient;
a shoulder harness, the shoulder harness having releasable straps for securing the shoulder harness to the body of the patient;
at least one head strap adjustably connected to the cap element such that a downward force is placed on the head of the patient;
at least one adjustable member operatively connected to the cap element and the shoulder harness, the adjustable member having a plurality of distances and a plurality of angles between the cap element and the shoulder harness.

2. The trauma cervical stability device of claim 1, wherein the cap element covers the posterior surface, the crown surface, and a forehead of the head of the patient.

3. The trauma cervical stability device of claim 1, wherein the cap element includes at least two portions, one of the at least two portions comprising a posterior portion and another of the at least two portions comprising an anterior portion.

4. The trauma cervical stability device of claim 1, wherein the trauma cervical stability device comprises at least two adjustable members operatively connected to the cap element and the shoulder harness, each of the adjustable members having a plurality of distances and a plurality of angles between the cap element and the shoulder harness.

5. The trauma cervical stability device of claim 5, wherein each of the at least two adjustable members each comprise an upper end and a lower end, each of the upper ends being rotatably connected to the cap element and each of the lower ends being operatively associated with a corresponding track disposed on the shoulder harness.

6. The trauma cervical stability device of claim 5, wherein the track includes an inner wall surface having a ratchet profile.

7. The trauma cervical stability device of claim 1, wherein the shoulder harness comprises releasable and adjustable straps for securing the shoulder harness to the body of the patient and the cap element comprises at least one head strap that is releasably and adjustably connected to the cap element.

8. The trauma cervical stability device of claim 1, further comprising a chin strap releasably and adjustably connected to the cap element.

9. The trauma cervical stability device of claim 8, wherein the at least two adjustable members are adjustably connected to the cap element and the shoulder harness and wherein at least two anterior adjustable members are adjustably connected to the chin strap and the shoulder harness, each of the at least two anterior adjustable members having a plurality of anterior distances and a plurality of anterior angles between the chin strap and the shoulder harness.

10. A method of diagnosing the severity of damage to a neck of a patient having a head and a body, the method comprising the steps of:
(a) securing a trauma cervical stability device to the neck of the patient to stabilize the head of the patient relative to the body of the patient, the neck having a plurality of vertebrae;
(b) applying to the trauma cervical stability device and, thus, to the head of the patient, a first known force in a first direction;
(c) measuring a first amount of movement of a first vertebrae of the patient in the first direction; and
(d) comparing the first amount of movement of the first vertebrae of the patient in the first direction to a first known amount of movement of the first vertebrae of the patient in the first direction to diagnose the severity of damage to the neck of the patient based upon whether the first amount of movement of the first vertebrae of the patient in the first direction is less than, equal to, or greater than the first known amount of movement of the first vertebrae of the patient in the first direction.

11. The method of claim 10, wherein at least one additional known force is applied to the trauma cervical stability device and, thus, to the first vertebrae of the patient, in at least one additional direction to measure at least one additional amount of movement of the first vertebrae of the patient in each of the at least one additional directions, and each of the at least one additional amounts of movement of the first vertebrae of the patient in each of the at least one additional directions is compared to corresponding known amounts of movement of the first vertebrae of the patient in each of the at least one additional directions to diagnose the severity of damage to the neck of the patient based upon whether each of the additional amounts of movement of the first vertebrae of the patient in each of the at least one additional directions is less than, equal to, or greater than the corresponding known amounts of movement of the first vertebrae of the patient in each of the at least one additional directions.

12. The method of claim 10, further comprising the steps of:
(e) applying to the trauma cervical stability device and, thus, to the head of the patient, a second known force in a second direction;
(f) measuring a second amount of movement of a second vertebrae of the patient in the second direction; and
(g) comparing the second amount of movement of the second vertebrae of the patient in the second direction to a second known amount of movement of the second vertebrae of the patient in the second direction to diagnose the severity of damage to the neck of the patient based upon whether the second amount of movement of the second vertebrae of the patient in the second direction is less than, equal to, or greater than the second known amount of movement of the second vertebrae of the patient in the second direction.

13. The method of claim 12, further comprising the steps of:
(h) applying to the trauma cervical stability device and, thus, to the head of the patient, a third known force in a third direction;
(i) measuring a third amount of movement of either the first vertebrae, the second vertebrae, or the first and second vertebra of the patient in the third direction; and
(j) comparing the third amount of movement of the first vertebrae, the second vertebrae, or the first and second vertebra of the patient in the third direction to a third known amount of movement of the first vertebrae, the second vertebrae, or the first and second vertebra of the patient in the third direction to diagnose the severity of damage to the neck of the patient based upon whether the third amount of movement of the first vertebrae, the second vertebrae, or the first and second vertebra of the patient in the second direction is less than, equal to, or greater than the third known amount of movement of the first vertebrae, the second vertebrae, or the first and second vertebra of the patient in the third direction.

14. The method of claim 13, wherein at least one additional known force is applied to the trauma cervical stability device and, thus, to the head of the patient, in at least one additional direction to measure at least one additional amount of movement of at least one vertebrae of the patient in each of the at least one additional directions, and each of the at least one additional amounts of movement of the at least one vertebrae of the patient in each of the at least one additional directions is compared to corresponding known amounts of movement of the at least one vertebrae of the patient in each of the at least one additional directions to diagnose the severity of damage to the neck of the patient based upon whether each of the additional amounts of movement of the at least one vertebrae of the patient in each of the at least one additional directions is less than, equal to, or greater than the corresponding known amounts of movement of the at least one vertebrae of the patient in each of the at least one additional directions.

15. The method of claim 10, wherein the severity of damage to the neck of a patient is diagnosed based upon the greater the first amount of movement of the first vertebrae of the patient in the first direction is as compared to the first known amount of movement in the first direction.

16. The method of claim 10, wherein the first known amount of movement of the first vertebrae of the patient in the first direction equals an amount of movement of the first vertebrae of the patient having an undamaged neck.

17. The method of claim 10, wherein the first known amount of movement of the first vertebrae of the patient in the first direction equals an amount of movement of a previously measured amount of movement of the first vertebrae of the patient having a damaged neck thereby allowing a determination as to whether the damaged neck is healing.

18. A method of determining the stability of a neck of a patient in relation to a spine of the patient, the method comprising the steps of:
(a) securing a trauma cervical stability device to the neck of the patient to stabilize a head of the patient relative to a body of the patient;
(b) applying a first load to the trauma cervical stability device and, thus, to the head of the patient, in a first direction;
(c) measuring a first amount of movement of a first vertebrae of the patient in the first direction; and
(d) comparing the first amount of movement of the first vertebrae of the patient in the first direction to a first known amount of movement of the first vertebrae of the patient in the first direction to determine whether the neck of the patient is stable,
wherein a difference between the first known amount of movement of the first vertebrae of the patient in the first direction and the first amount of movement of the first vertebrae of the patient in the first direction indicates that the neck is not stable.

19. The method of claim 18, wherein at least one additional known load is applied to the trauma cervical stability device and, thus, to the head of the patient, in at least one additional direction to measure at least one additional amount of movement of at least one vertebrae of the patient in each of the at least one additional directions, and each of the at least one additional amounts of movement of the at least one vertebrae of the patient in each of the at least one additional directions is compared to corresponding known amounts of movement of the at least one vertebrae of the patient in each of the at least one additional directions to determine whether the neck of the patient is stable,
wherein a difference between one or more of the at least one of additional amounts of movement of the at least one vertebrae of the patient in each of the at least one additional directions and the corresponding known amounts of movement of the at least one vertebrae of the patient in each of the at least one additional directions indicates that the neck is not stable.

20. The method of claim 18, wherein the first load is applied to the trauma cervical stability device and, thus, to the head of the patient, in a first direction by affixing a known weight to the trauma cervical stability device.

21. The method of claim 20, the first known amount of movement of the at least one vertebrae of the patient in the first direction is based upon the known weight, and the first known amount of movement of the at least one vertebrae of the patient in the first direction is known prior to applying the first load.
